(19) United States
(12) Patent Application Publication
(10) Pub. No.: US 2022/0409774 A1
(43) Pub. Date: Dec. 29, 2022

(54) ERYTHROPOIETIN COMPOSITIONS AND METHODS OF USE

(71) Applicant: The Penn State Research Foundation, University Park, PA (US)

(72) Inventors: Kristen Manto, Hummelstown, PA (US); John Elfar, Hummelstown, PA (US)

(21) Appl. No.: 17/728,582

(22) Filed: Apr. 25, 2022

Related U.S. Application Data
(60) Provisional application No. 63/182,403, filed on Apr. 30, 2021.

(51) Int. Cl. A61L 27/52; A61P 17/02; A61K 9/06; C07K 14/575

(52) U.S. Cl. CPC A61L 27/52; A61P 17/02; A61K 9/06; C07K 14/575

(57) ABSTRACT

This document relates to compositions containing one or more erythropoietin (EPO) polypeptides. For example, this document provides thermoresponsive compositions containing one or more EPO polypeptides and methods for using such thermoresponsive compositions as a delivery system to deliver one or more EPO polypeptides to desired tissue (e.g., to treat a nerve injury and/or a wound). In some cases, thermoresponsive compositions containing one or more EPO polypeptides can be administered (e.g., locally administered) to a mammal having a nerve injury to treat the nerve injury (e.g., to promote wound healing). In some cases, thermoresponsive compositions containing one or more EPO polypeptides can be administered (e.g., locally administered) to a mammal having a wound to treat the wound (e.g., to promote wound healing).

### CROSS-REFERENCE TO RELATED APPLICATIONS

This application claims the benefit of U.S. patent application Ser. No. 63/182,403, filed on Apr. 30, 2021. The disclosure of the prior application is considered part of (and is incorporated by reference in) the disclosure of this application.

### STATEMENT REGARDING FEDERAL FUNDING

This invention was made with government support under Grant No. AR060164 awarded by the National Institutes of Health and under Grant No. W81XWH-16-1-0725 awarded by the United States Army/MRMC. The Government has certain rights in the invention.

### BACKGROUND

### 1. Technical Field

This document relates to compositions containing one or more erythropoietin (EPO) polypeptides. For example, this document provides thermoresponsive compositions containing one or more EPO polypeptides and methods for using such thermoresponsive compositions as a delivery system to deliver one or more EPO polypeptides to a desired tissue (e.g., to treat a nerve injury and/or a wound). In some cases, thermoresponsive compositions containing one or more EPO polypeptides can be administered (e.g., locally administered) to a mammal having a nerve injury to treat the nerve injury (e.g., to promote wound healing). In some cases, thermoresponsive compositions containing one or more EPO polypeptides can be administered (e.g., locally administered) to a mammal having a wound to treat the wound (e.g., to promote wound healing).

### 2. Background Information

EPO stimulates red blood cell production (erythropoiesis) in the bone marrow, and can be used to treat many indications. However, current efforts at developing locally applicable formulations of EPO have been nanoparticle based, and have lacked adherence properties for targeted delivery to an injury site.

### SUMMARY

This document provides compositions containing one or more EPO polypeptides and methods for using compositions containing one or more EPO polypeptides. For example, this document provides thermoresponsive compositions (e.g., thermoresponsive polymer compositions) containing one or more EPO polypeptides. In some cases, a thermoresponsive composition described herein can be used as a delivery system to deliver one or more EPO polypeptides to a desired tissue or injury. For example, a thermoresponsive composition containing one or more EPO polypeptides can be administered (e.g., locally administered) to a mammal in need thereof (e.g., a mammal having a nerve injury and/or having a wound such as a skin wound or an internal wound). In some cases, a thermoresponsive composition described herein can be used to treat an injured nerve (e.g., an injured nerve within a mammal). For example, a thermoresponsive composition described herein can be delivered onto, into, around, and/or near an injured nerve (e.g., a nerve injury site) within a mammal to treat the injured nerve. In some cases, a thermoresponsive composition described herein can be used to treat a wound (e.g., a skin wound or an internal wound). For example, a thermoresponsive composition described herein can be delivered onto, into, around, and/or near a wound (e.g., a skin wound or an internal wound) within a mammal to treat the wound (e.g., to promote wound healing).

As demonstrated herein, one or more EPO polypeptides can be formulated into a thermoresponsive composition containing block poly lactic-co-glycolic acid (PLGA) (e.g., a copolymer of poly lactic acid (PLA) and poly glycolic acid (PGA) such as poly(lactide-co-glycolide)-b-poly(ethylene glycol)-b-poly(lactide-co-glycolide)) and polyethylene glycol (PEG) that is a liquid at room temperature (e.g., at about 25° C.) and undergoes a phase transition to form a gel at body temperature (e.g., at about 37° C. for a human). Such a thermoresponsive composition can be injected as a liquid into a mammal (e.g., can be injected onto, into, around, and/or near site in need of treatment with one or more EPO polypeptides) and can form a hydrogel in situ at the injection site. Also as demonstrated herein, a thermoresponsive EPO-PLGA-PEG composition can be used for controlled-release of one or more EPO polypeptides and can deliver one or more EPO polypeptides onto, into, around, and/or near the injection site for up to several weeks at a certain sustained rate of release.

Having the ability to deliver one or more EPO polypeptides directly to an injury (e.g., a nerve injury and/or a wound) using a temperature-sensitive, controlled-release formulation allows clinicians to provide patients with local and controlled treatments of injuries. Local and controlled delivery of one or more EPO polypeptides provides a non-invasive treatment option that can bypass the need for unnecessary surgical intervention and provide targeted treatment. Moreover, local and controlled delivery of one or more EPO polypeptides avoids the potential adverse side effects seen with systemic dosing of EPO polypeptides in humans.

In general, one aspect of this document features thermoresponsive compositions including: (a) a thermoresponsive polymer comprising PLGA and PEG, and (b) an EPO polypeptide or a variant of the EPO polypeptide. The thermoresponsive composition can be a liquid when below a physiological temperature of a mammal and can be a gel when at or above the physiological temperature of the mammal. The thermoresponsive composition can be a liquid at about 20° C. to about 31° C. The thermoresponsive composition can be a gel at about 37° C. The PLGA can have a molecular weight of about 200 Daltons (Da) to about 75,000 Da. The PEG can have a molecular weight of about 200 Da to about 75,000 Da. The polymer can include from about 0.04:1 PLGA:PEG to about 32:1 of PLGA:PEG The thermoresponsive composition can include from about 0.08 M to about 4 M of the EPO polypeptide or the variant of the EPO polypeptide.

In another aspect, this document features methods for treating a nerve injury within a mammal. The methods can include, or consist essentially of, administering a thermoresponsive composition including: (a) a thermoresponsive polymer comprising PLGA and PEG, and (b) an EPO polypeptide or a variant of the EPO polypeptide onto, into, around, and/or near a nerve injury within a mammal. The mammal can be a human. The thermoresponsive composition can be a liquid when below a physiological temperature of the mammal and can be a gel when at or above the physiological temperature of the mammal such that the thermoresponsive composition, once administered onto, into, around, and/or near the nerve injury forms a gel in situ within the mammal. The administration can include an injection. The gel can release about 0.005 mg/kg to about 5 mg/kg of the EPO polypeptide or the variant of the EPO polypeptide. The gel can release the EPO polypeptide or the variant of the EPO polypeptide for about 1 minute to about 4 weeks.

In another aspect, this document features methods for treating a wound within a mammal. The methods can include, or consist essentially of, administering a thermoresponsive composition including: (a) a thermoresponsive polymer comprising PLGA and PEG; and (b) an EPO polypeptide or a variant of the EPO polypeptide onto, into, around, and/or near a wound within a mammal. The mammal can be a human. The wound can be a cutaneous wound. The administration can include an injection.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A and 1B contain graphs showing in vitro release of EPO polypeptides from a PLGA-PEG hydrogel (AK092; 3:1 LA:GA). FIG. 1A: Cumulative EPO release from a PLGA-PEG hydrogel (3:1 LA:GA) over 24 hours. FIG. 1B: Cumulative EPO release from a PLGA-PEG hydrogel (3:1 LA:GA) over 5 days.

FIG. 2 contains a graph showing cumulative in vitro release of EPO polypeptides from a PLGA-PEG hydrogel (AK097; 15:1 LA:GA).

FIGS. 3A and 3B contain graphs showing gelation temperature of an EPO-PLGA-PEG hydrogel. FIG. 3A: Dynamic temperature sweep from 10° C. to 40° C. FIG. 3B: Dynamic temperature sweep from 20° C. to 38° C.

FIGS. 4A and 4B contain graphs showing gelation temperature of an EPO-PLGA-PEG hydrogel. FIG. 4A: Dynamic temperature sweep from 10° C. to 40° C. FIG. 4B: Dynamic temperature sweep from 20° C. to 38° C.

FIGS. 5A and 5B contain graphs showing gelation time of hydrogels. FIG. 5A: Dynamic time sweep to determine time evolution of viscoelastic response from 25° C. to 37° C. of a PLGA-PEG hydrogel. FIG. 5B: Dynamic temperature sweep to determine time evolution of viscoelastic response from 25° C. to 37° C. of an EPO-PLGA-PEG hydrogel.

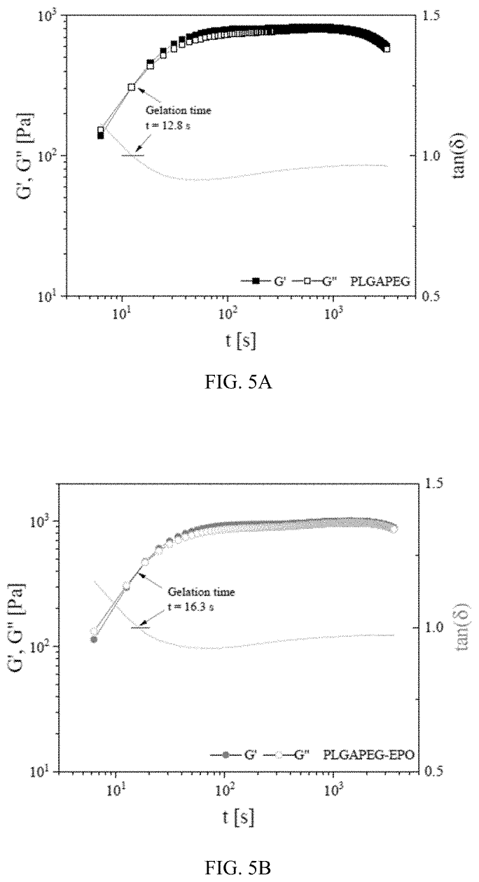

FIGS. 6A and 6B contain graphs showing that solution-gel transition of hydrogels is reversible. FIG. 6A: Dynamic time sweep to determine reversibility from 37° C. to 25° C. of a PLGA-PEG hydrogel. FIG. 6B: Dynamic time sweep to determine reversibility from 37° C. to 25° C. of an EPO-PLGA-PEG hydrogel.

FIGS. 7A and 7B contain graphs showing hemoglobin (FIG. 7A) and hematocrit (FIG. 7B) after administering an EPO-PLGA-PEG hydrogel.

FIGS. 8A and 8B contains graphs showing grip strength (FIG. 8A) and response to stimulus (FIG. 8B) after administering an EPO-PLGA-PEG hydrogel.

FIG. 9 contains images showing in vivo biodegradation of (EPO)-PLGA-PEG and local neovascularization.

FIG. 10 contains an amino acid sequence (SEQ ID NO:1) of an exemplary human EPO polypeptide.

### DETAILED DESCRIPTION

This document provides compositions containing one or more EPO polypeptides and methods for using compositions containing one or more EPO polypeptides. For example, this document provides thermoresponsive compositions (e.g., thermoresponsive polymer compositions) containing one or more EPO polypeptides. In some cases, a thermoresponsive composition described herein can be used as a delivery system to deliver one or more EPO polypeptides to a desired tissue (e.g., a wound). For example, a thermoresponsive composition containing one or more EPO polypeptides can be administered (e.g., locally administered) to a mammal in need thereof (e.g., a mammal having a nerve injury and/or having a wound such as a skin wound or an internal wound). In some cases, a thermoresponsive composition described herein can be used to treat an injured nerve (e.g., an injured nerve within a mammal). For example, a thermoresponsive composition described herein can be delivered onto, into, around, and/or near an injured nerve (e.g., a nerve injury site) within a mammal to treat the injured nerve. In some cases, a thermoresponsive composition described herein can be used to treat a wound (e.g., a skin wound or an internal wound). For example, a thermoresponsive composition described herein can be delivered onto, into, around, and/or near a wound (e.g., a skin wound or an internal wound) within a mammal to treat the wound (e.g., to promote wound healing).

A thermoresponsive composition described herein (e.g., a thermoresponsive polymer composition containing one or more EPO polypeptides) can be a three-dimensional network of polymeric chains that can change phases (e.g., from a liquid phase to a gel phase or vice versa) as the temperature changes. In some cases, a thermoresponsive composition can be a liquid at a lower temperature such as an ambient temperature (e.g., at room temperature such as about 22° C.) and can transition to a gel at a higher temperature such as a physiological temperature (e.g., at body temperature such as 37° C.). For example, a thermoresponsive composition can be a liquid at a temperature of from about 20° C. to about 31° C. (e.g., from about 20° C. to about 30° C., from about 20° C. to about 28° C., from about 20° C. to about 25° C., from about 20° C. to about 22° C., from about 23° C. to about 31° C., from about 25° C. to about 31° C., from about 27° C. to about 31° C., from about 30° C. to about 31° C., from about 22° C. to about 30° C., from about 23° C. to about 28° C., from about 22° C. to about 25° C., or from about 25° C. to about 28° C.). In some cases, the phase transition of a thermoresponsive composition described herein can be reversible. In some cases, the phase transition of a thermoresponsive composition described herein can be irreversible. The temperature at or below which a thermoresponsive composition can be maintained in a liquid phase can be referred to as the lower critical solution temperature (LCST). A thermoresponsive composition described herein (e.g., a thermoresponsive polymer composition containing one or more EPO polypeptides) can have any appropriate LCST. In some cases, a LCST of a thermoresponsive composition described herein can be an ambient temperature (e.g., room temperature). For example, a LCST of a thermoresponsive composition described herein can be from about 30.9° C. to about 37° C. (e.g., from about 30.9° C. to about 35° C., from about 30.9° C. to about 32° C., from about 32° C. to about 37° C., from about 35° C. to about 37° C., from about 31° C. to about 35° C., from about 32° C. to about 34° C., from about 31° C. to about 33° C., or from about 33° C. to about 35° C.). The temperature at which a thermoresponsive composition can change phases (e.g., from a liquid phase to a gel phase or vice versa) can be referred to as the transition temperature. A thermoresponsive composition described herein (e.g., a thermoresponsive polymer composition containing one or more EPO polypeptides) can have any appropriate transition temperature. In some cases, a transition temperature of a thermoresponsive composition described herein can be a physiological temperature (e.g., body temperature of a mammal). For example, a transition temperature of a thermoresponsive composition described herein can be from about 30.9° C. to about 37° C. (e.g., from about 30.9° C. to about 35° C., from about 30.9° C. to about 32° C., from about 32° C. to about 37° C., from about 35° C. to about 37° C., from about 31° C. to about 35° C., from about 32° C. to about 34° C., from about 31° C. to about 33° C., or from about 33° C. to about 35° C.). The LCST and/or the transition temperature of a thermoresponsive composition described herein can be affected by many structural parameters of the thermoresponsive composition such as the hydrophobic content, architecture of the thermoresponsive composition, molar mass of the thermoresponsive composition, the relative ratio of hydrophobic to hydrophilic components in the thermoresponsive composition, and any combinations thereof.

In some cases, a thermoresponsive composition described herein (e.g., a thermoresponsive polymer composition one or more EPO polypeptides) can be biodegradable. For example, a thermoresponsive composition described herein can be decomposed by a living organism (e.g., by a biological process).

In some cases, a thermoresponsive composition described herein (e.g., a thermoresponsive polymer composition containing one or more EPO polypeptides) can be non-biodegradable.

In some cases, a thermoresponsive composition described herein (e.g., a thermoresponsive polymer composition containing one or more EPO polypeptides) can be biocompatible. For example, a thermoresponsive composition described herein can produce minimal to no adverse effects (e.g., toxicity, irritation, allergies, and/or rejection) when administered to a mammal (e.g., a human).

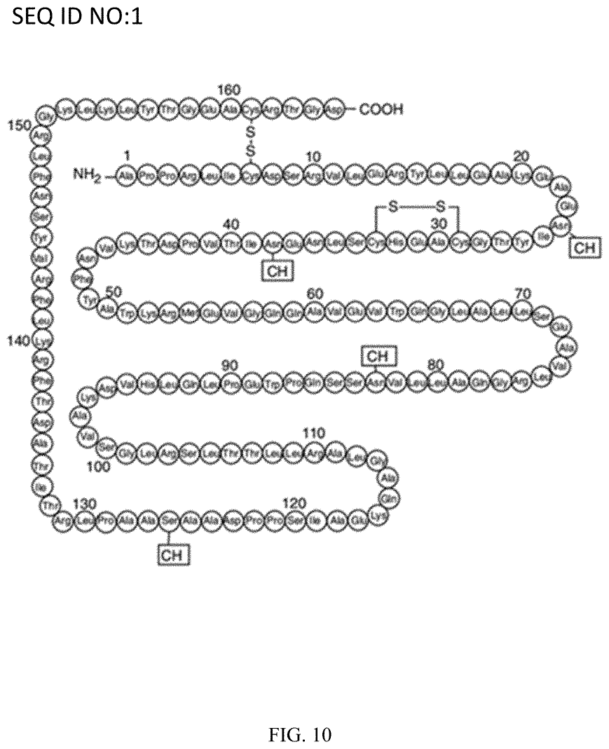

A thermoresponsive composition described herein (e.g., a thermoresponsive polymer composition containing one or more EPO polypeptides) can include any appropriate EPO polypeptide(s). Examples of EPO polypeptides that can be used as described herein include, without limitation, EPO polypeptides having the amino acid sequence set forth in National Center for Biotechnology Information (NCBI) under Gene ID: 2056, GenPept® Accession No. P01588, GenPept® Accession No. AAF23132, GenPept® Accession No. AAF23134, and GenPept® Accession No. AAI11938. In some cases, an EPO polypeptide can have an amino acid sequence set forth in SEQ ID NO:1 (see, e.g., FIG. 10).

In some cases, a variant of an EPO polypeptide can be used in place of or in addition to an EPO polypeptide. A variant of an EPO polypeptide can have the amino acid sequence of a naturally-occurring EPO polypeptide with one or more (e.g., one, two, three, four, five, six, seven, eight, nine, ten, or more) amino acid deletions, additions, substitutions, or combinations thereof, provided that the variant retains the function of a naturally-occurring EPO polypeptide (e.g., provided that the variant can bind the an EPO receptor to stimulate red blood cell production and/or stimulate angiogenesis).

Any appropriate amino acid residue set forth in SEQ ID NO:1 can be deleted, and any appropriate amino acid residue (e.g., any of the 20 conventional amino acid residues or any other type of amino acid such as ornithine or citrulline) can be added to or substituted within the sequence set forth in SEQ ID NO:1. The majority of naturally occurring amino acids are L-amino acids, and naturally occurring polypeptides are largely comprised of L-amino acids. D-amino acids are the enantiomers of L-amino acids. In some cases, a polypeptide provided herein can contain one or more D-amino acids. In some embodiments, a polypeptide can contain chemical structures such as ε-aminohexanoic acid; hydroxylated amino acids such as 3-hydroxyproline, 4-hydroxyproline, (5R)-5-hydroxy-L-lysine, allo-hydroxylysine, and 5-hydroxy-L-norvaline; or glycosylated amino acids such as amino acids containing monosaccharides (e.g., D-glucose, D-galactose, D-mannose, D-glucosamine, and D-galactosamine) or combinations of monosaccharides.

Amino acid substitutions can be made, in some cases, by selecting substitutions that do not differ significantly in their effect on maintaining (a) the structure of the peptide backbone in the area of the substitution, (b) the charge or hydrophobicity of the molecule at particular sites, or (c) the bulk of the side chain. For example, naturally occurring residues can be divided into groups based on side-chain properties: (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of substitutions that can be used herein for SEQ ID NO:1 include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine. Further examples of conservative substitutions that can be made at any appropriate position within SEQ ID NO:1 are set forth in Table 1 below.

TABLE 1
Examples of conservative amino acid substitutions.

| Original | | Preferred |
|---|---|---|
| Residue | Exemplary substitutions | substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn | Asn |
| Glu | Asp | Asp |
| Gly | Pro | Pro |
| His | Asn, Gln, Lys, Arg | Arg |
| Ile | Leu, Val, Met, Ala, Phe, Norleucine | Leu |
| Leu | Norleucine, Ile, Val, Met, Ala, Phe | He |
| Lys | Arg, Gln, Asn | Arg |
| Met | Leu, Phe, Ile | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleucine | Leu |

In some cases, a variant of an EPO polypeptide can be designed to include the amino acid sequence set forth in SEQ ID NO:1 with the proviso that it includes one or more non-conservative substitutions. Non-conservative substitutions typically entail exchanging a member of one of the classes described above for a member of another class. Whether an amino acid change results in a functional polypeptide can be determined by assaying the specific activity of the polypeptide using, for example, the methods described herein.

In some cases, a variant of an EPO polypeptide having an amino acid sequence with at least 85% (e.g., 85%, 86%, 87%, 88%, 89%, 90%, 91%, 92%, 93%, 94%, 95%, 96%, 97%, 98%, or 99.0%) sequence identity to the amino acid sequence set forth in SEQ ID NO:1, provided that it includes at least one difference (e.g., at least one amino acid addition, deletion, or substitution) with respect to SEQ ID NO:1, can be used. Percent sequence identity is calculated by determining the number of matched positions in aligned amino acid sequences, dividing the number of matched positions by the length of an aligned amino acid sequence, and multiplying by 100. A matched position refers to a position in which identical amino acids occur at the same position in aligned amino acid sequences. Percent sequence identity also can be determined for any nucleic acid sequence.

The percent sequence identity between a particular nucleic acid or amino acid sequence and a sequence referenced by a particular sequence identification number (e.g., SEQ ID NO:1) is determined as follows. First, a nucleic acid or amino acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: C:\B12seq -i c:\seq1.txt -j c:\seq2.txt -p blastn -o c:\output.txt -q −1-r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches by the length of the alignment, followed by multiplying the resulting value by 100. For example, an amino acid sequence that has 340 matches when aligned with the sequence set forth in SEQ ID NO:1 is 95.5 percent identical to the sequence set forth in SEQ ID NO:1 (i.e., 340±356×100=95.5056). It is noted that the percent sequence identity value is rounded to the nearest tenth. For example, 75.11, 75.12, 75.13, and 75.14 is rounded down to 75.1, while 75.15, 75.16, 75.17, 75.18, and 75.19 is rounded up to 75.2. It also is noted that the length value will always be an integer.

In some cases, an EPO polypeptide (or a variant of an EPO polypeptide) that can be included in a thermoresponsive composition described herein (e.g., a thermoresponsive polymer composition containing one or more EPO polypeptides) can be as described elsewhere (see, e.g., GenScript® Cat. No. Z02975).

A thermoresponsive composition described herein (e.g., a thermoresponsive polymer composition containing one or more EPO polypeptides) can include any appropriate amount of one or more EPO polypeptides. In some cases, a thermoresponsive composition described herein can include from about 0.08 M to about 4 M (e.g., from about 0.08 M to about 3.5 M, from about 0.08 M to about 3 M, from about 0.08 M to about 2.5 M, from about 0.08 M to about 2 M, from about 0.08 M to about 1.5 M, from about 0.08 M to about 1 M, from about 0.08 M to about 0.5 M, from about 0.5 M to about 4 M, from about 1 M to about 4 M, from about 1.5 M to about 4 M, from about 2 M to about 4 M, from about 2.5 M to about 4 M, from about 3 M to about 4 M, from about 3.5 M to about 4 M, from about 0.5 M to about 3.5 M, from about 1 M to about 3 M, from about 1.5 M to about 2.5 M, from about 0.5 M to about 1.5 M, or from about 2.5 M to about 3.5 M) of one or more EPO polypeptides. In some cases, a thermoresponsive composition described herein can include from about 0.01 mg/mL to about 10 mg/mL (e.g., from about 0.01 mg/mL to about 9 mg/mL, from about 0.01 mg/mL to about 8 mg/mL, from about 0.01 mg/mL to about 7 mg/mL, from about 0.01 mg/mL to about 6 mg/mL, from about 0.01 mg/mL to about 5 mg/mL, from about 0.01 mg/mL to about 4 mg/mL, from about 0.01 mg/mL to about 3 mg/mL, from about 0.01 mg/mL to about 2 mg/mL, from about 0.01 mg/mL to about 1 mg/mL, from about 0.01 mg/mL to about 0.1 mg/mL, from about 0.1 mg/mL to about 10 mg/mL, from about 1 mg/mL to about 10 mg/mL, from about 2 mg/mL to about 10 mg/mL, from about 3 mg/mL to about 10 mg/mL, from about 4 mg/mL to about 10 mg/mL, from about 5 mg/mL to about 10 mg/mL, from about 6 mg/mL to about 10 mg/mL, from about 7 mg/mL to about 10 mg/mL, from about 8 mg/mL to about 10 mg/mL, from about 9 mg/mL to about 10 mg/mL, from about 0.1 mg/mL to about 9 mg/mL, from about 1 mg/mL to about 8 mg/mL, from about 2 mg/mL to about 7 mg/mL, from about 3 mg/mL to about 6 mg/mL, from about 4 mg/mL to about 5 mg/mL, from about 0.1 mg/mL to about 2 mg/mL, from about 1 mg/mL to about 3 mg/mL, from about 3 mg/mL to about 5 mg/mL, from about 5 mg/mL to about 7 mg/mL, or from about 7 mg/mL to about 9 mg/mL) of one or more EPO polypeptides. In some cases, a thermoresponsive composition described herein can include from about 1% to about 50% (e.g., from about 1% to about 40%, from about 1% to about 30%, from about 1% to about 20%, from about 1% to about 10%, from about 10% to about 50%, from about 20% to about 50%, from about 30% to about 50%, from about 40% to about 50%, from about 5% to about 45%, from about 10% to about 40%, from about 15% to about 35%, from about 20% to about 30%, from about 5% to about 15%, from about 15% to about 25%, from about 25% to about 35%, or from about 35% to about 45%) of one or more EPO polypeptides.

A thermoresponsive composition described herein (e.g., a thermoresponsive polymer composition containing one or more EPO polypeptides) can include any appropriate polymer(s). Examples of polymers that can be included in a thermoresponsive composition described herein include, without limitation, PLGA, PEG PLLA, PCL, PLCL, PDLLA, PLA, mPEG PVL, PTMC, TEG PEI, P(DL)LA, P(D)La, PGA, and any combinations thereof. A polymer can have any appropriate molecular weight (MW; e.g., an average MW). For example, a polymer that can be included in a thermoresponsive composition described herein can have a molecular weight of from about 72 Daltons (Da) to about 75,000 Da (e.g., from about 72 Da to about 70,000 Da, from about 72 Da to about 60,000 Da, from about 72 Da to about 50,000 Da, from about 72 Da to about 40,000 Da, from about 72 Da to about 30,000 Da, from about 72 Da to about 20,000 Da, from about 72 Da to about 15,000 Da, from about 72 Da to about 10,000 Da, from about 72 Da to about 5,000 Da, from about 72 Da to about 1,000 Da, from about 72 Da to about 500 Da, from about 200 Da to about 75,000 Da, from about 500 Da to about 75,000 Da, from about 1,000 Da to about 75,000 Da, from about 5,000

Da to about 75,000 Da, from about 10,000 Da to about 75,000 Da, from about 20,000 Da to about 75,000 Da, from about 30,000 Da to about 75,000 Da, from about 40,000 Da to about 75,000 Da, from about 50,000 Da to about 75,000 Da, from about 60,000 Da to about 75,000 Da, from about 100 Da to about 50,000 Da, from about 200 Da to about 25,000 Da, from about 300 Da to about 15,000 Da, from about 400 Da to about 10,000 Da, from about 100 Da to about 1,000 Da, from about 1,000 Da to about 5,000 Da, from about 5,000 Da to about 10,000 Da, from about 10,000 Da to about 25,000 Da, or from about 25,000 Da to about 50,000 Da). For example, when a polymer is a PLGA, the PLGA can have a MW (e.g., an average MW) of from about 200 Da to about 75,000 Da (e.g., from about 200 Da to about 65,000 Da, from about 200 Da to about 50,000 Da, from about 200 Da to about 35,000 Da, from about 200 Da to about 20,000 Da, from about 200 Da to about 10,000 Da, from about 200 Da to about 5,000 Da, from about 200 Da to about 1,000 Da, from about 200 Da to about 500 Da, from about 500 Da to about 75,000 Da, from about 1,000 Da to about 75,000 Da, from about 1,500 Da to about 75,000 Da, from about 20,000 Da to about 75,000 Da, from about 45,000 Da to about 75,000 Da, from about 500 Da to about 50,000 Da, from about 10,000 Da to about 25,000 Da, from about 15,000 Da to about 20,000 Da, from about 500 Da to about 1,000 Da, from about 1,000 Da to about 2,000 Da, from about 2,000 Da to about 25,000 Da, or from about 25,000 Da to about 50,000 Da). In some cases, when a polymer is a PLGA, the PLGA can have a MW of from about 1,500 Da to about 2,000 Da (e.g., about 1,700 Da). For example, when a polymer is a PEG, the PEG can have a MW (e.g., an average MW) of from about 400 Da to about 10,000 Da (e.g., from about 400 Da to about 8,000 Da, from about 400 Da to about 5,000 Da, from about 400 Da to about 3,000 Da, from about 400 Da to about 2,000 Da, from about 400 Da to about 1,000 Da, from about 1,000 Da to about 10,000 Da, from about 2,000 Da to about 10,000 Da, from about 3,000 Da to about 10,000 Da, from about 5,000 Da to about 10,000 Da, from about 8,000 Da to about 10,000 Da, from about 500 Da to about 8,000 Da, from about 1,000 Da to about 5,000 Da, from about 2,000 Da to about 4,000 Da, from about 500 Da to about 1,000 Da, from about 1,000 Da to about 3,000 Da, from about 3,000 Da to about 5,000 Da, from about 5,000 Da to about 7,000 Da, or from about 7,000 Da to about 9,000 Da). In some cases, when a polymer is a PEG, the PEG can have a MW of from about 1,000 Da to about 2,000 Da (e.g., about 1,500 Da). Unless otherwise specified, polymer MWs provided herein are weight average MW. In some cases, a polymer can be a copolymer (e.g., can be formed from polymerization of two or more different monomers). When a polymer is a copolymer, the copolymer can be any type of copolymer (e.g., a linear copolymer or a branched copolymer). Examples of types of copolymers that can be used as described herein include, without limitation, alternating copolymers, statistical copolymers, gradient copolymers, block copolymers, and grafted copolymers. A polymer can be a natural polymer or a synthetic polymer. In some cases, a polymer can be a biodegradable polymer. In some cases, a polymer can be a biocompatible polymer. In some cases, a polymer included in a thermoresponsive composition described herein can include one or more functional groups (e.g., hydrophilic functional groups) in its polymeric structures. Examples of functional groups that can be included in a polymer of a thermoresponsive composition described herein include, without limitation, amino groups (e.g., NH₂), hydroxyl groups (e.g., OH), amide groups (e.g. CONH— and CONH₂), and sulfate groups (e.g., —SO₃H).

In some cases, a thermoresponsive composition described herein (e.g., a thermoresponsive polymer composition containing one or more EPO polypeptides) can be a copolymer. A copolymer can include any appropriate ratio of a first polymer to a second polymer. For example, when a thermoresponsive composition described herein includes a PLGA copolymer, the PLGA can include from about 3:1 LA:GA to about 15:1 of LA:GA (e.g., from about 3:1 LA:GA to about 12:1 of LA:GA, from about 3:1 LA:GA to about 10:1 of LA: GA, from about 3:1 LA: GA to about 8:1 of LA: GA, from about 3:1 LA: GA to about 5:1 of LA: GA, from about 5:1 LA: GA to about 15:1 of LA: GA, from about 7:1 LA: GA to about 15:1 of LA:GA, from about 10:1 LA:GA to about 15:1 of LA:GA, from about 12:1 LA:GA to about 15:1 of LA:GA, from about 5:1 LA:GA to about 10:1 of LA: GA, from about 4:1 LA:GA to about 8:1 of LA:GA, or from about 8:1 LA:GA to about 12:1 of LA:GA). In some cases, when a thermoresponsive composition described herein includes a PLGA copolymer, the PLGA can include about 3:1 LA:GA. In some cases, when a thermoresponsive composition described herein includes a PLGA copolymer, the PLGA can include about 15:1 LA:GA. For example, when a thermoresponsive composition described herein includes a PLGA-PEG copolymer (e.g., a PLGA-PEG block copolymer), the thermoresponsive composition described herein can include from about 0.04:1 PLGA:PEG to about 32:1 of PLGA:PEG (e.g., from about 0.5:1 PLGA:PEG to about 32:1 of PLGA:PEG from about 1:1 PLGA:PEG to about 32:1 of PLGA:PEG from about 5:1 PLGA:PEG to about 32:1 of PLGA:PEG, from about 10:1 PLGA:PEG to about 32:1 of PLGA:PEG, from about 15:1 PLGA:PEG to about 32:1 of PLGA:PEG from about 20:1 PLGA:PEG to about 32:1 of PLGA:PEG from about 25:1 PLGA:PEG to about 32:1 of PLGA:PEG from about 0.04:1 PLGA:PEG to about 30:1 of PLGA:PEG from about 0.04:1 PLGA:PEG to about 20:1 of PLGA:PEG from about 0.04:1 PLGA:PEG to about 15:1 of PLGA:PEG from about 0.04:1 PLGA:PEG to about 10:1 of PLGA:PEG from about 0.04:1 PLGA:PEG to about 5:1 of PLGA:PEG from about 0.04:1 PLGA:PEG to about 1:1 of PLGA:PEG from about 1:1 PLGA:PEG to about 25:1 of PLGA:PEG from about 5:1 PLGA:PEG to about 20:1 of PLGA:PEG from about 10:1 PLGA:PEG to about 15:1 of PLGA:PEG from about 1:1 PLGA:PEG to about 10:1 of PLGA:PEG from about 10:1 PLGA:PEG to about 20:1 of PLGA:PEG or from about 20:1 PLGA:PEG to about 30:1 of PLGA:PEG).

In some cases, polymers within a thermoresponsive composition described herein (e.g., a thermoresponsive polymer composition containing one or more EPO polypeptides) can be crosslinked or can be such that they are not crosslinked.

In some cases, a thermoresponsive composition described herein (e.g., a thermoresponsive polymer composition containing one or more EPO polypeptides) can include one or more EPO polypeptides and a PLGA-PEG copolymer (e.g., a PLGA-PEG block copolymer). For example, a thermoresponsive composition described herein can include from about 0.08 M of one or more EPO polypeptides to about 4 M of one or more EPO polypeptides, can include from about 65% to about 75% PLGA (e.g., about 69.3% PLGA), and can include from about 30% to about 40% PEG (e.g., about 34.7% PEG).

In some cases, a thermoresponsive composition described herein (e.g., a thermoresponsive polymer composition containing one or more EPO polypeptides) can include one or more pharmaceutically acceptable carriers (additives), excipients, and/or diluents. Examples of pharmaceutically acceptable carriers, excipients, and diluents that can be used in a thermoresponsive composition described herein include, without limitation, water, phosphate-buffered saline (PBS), sucrose, lactose, starch (e.g., starch glycolate), cellulose, cellulose derivatives (e.g., modified celluloses such as microcrystalline cellulose and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, and lecithin.

In some cases, a thermoresponsive composition described herein (e.g., a thermoresponsive polymer composition containing one or more EPO polypeptides) can be formulated as a delivery system to deliver EPO polypeptides (e.g., can be formulated to release EPO polypeptides from the thermoresponsive composition). For example, a thermoresponsive composition described herein can be formulated to release EPO polypeptides from the thermoresponsive composition when the thermoresponsive composition is in its gel phase. For example, a thermoresponsive composition described herein can be formulated as a controlled-release delivery system to deliver EPO polypeptides from the thermoresponsive composition. Examples of types of controlled-release delivery that a thermoresponsive composition described herein can be formulated for include, without limitation, burst release, slow release, delayed release, and sustained release. When a thermoresponsive composition is formulated for a sustained release of EPO polypeptides from the gel phase of the thermoresponsive composition, the sustained release can release the EPO polypeptides present in the thermoresponsive composition for from about 1 minute to about 4 weeks (e.g., from about 1 minute to about 3 weeks, from about 1 minute to about 2 weeks, from about 60 minutes to about 4 weeks, from about 1 hour to about 4 weeks, from about 1 day to about 4 weeks, from about 1 week to about 4 weeks, from about 1 day to about 3 weeks, from about 1 week to about 2 weeks, or from about 2 weeks to about 4 weeks) after administering the thermoresponsive composition to a mammal (e.g., after the thermoresponsive composition has formed a gel). In some cases, a thermoresponsive composition that is formulated for a sustained release of EPO polypeptides from the gel phase of the thermoresponsive composition can release the EPO polypeptides present in the thermoresponsive composition for from about 60 minutes to about 3 weeks after administering the thermoresponsive composition to a mammal (e.g., after the thermoresponsive composition has formed a gel). In some cases, a thermoresponsive composition described herein can be formulated to provide two or more types of controlled-release of EPO polypeptides from the thermoresponsive composition in its gel phase. For example, a thermoresponsive composition described herein can be formulated to provide a burst release of EPO polypeptides from the thermoresponsive composition in its gel phase followed by a sustained release of any remaining EPO polypeptides from the thermoresponsive composition in its gel phase.

This document also provides methods and materials for using a thermoresponsive composition described herein (e.g., a thermoresponsive polymer composition containing one or more EPO polypeptides). In some cases, a thermoresponsive composition described herein (e.g., an effective amount of a thermoresponsive composition containing one or more EPO polypeptides) can used for treating a mammal in need thereof (e.g., a mammal having a nerve injury and/or having a wound such as a skin wound or an internal wound). For example, a thermoresponsive composition described herein can be administered to a mammal having a nerve injury to treat the mammal. For example, a thermoresponsive composition described herein can be administered to a mammal having a wound to treat the mammal.

In some cases, a thermoresponsive composition described herein (e.g., a thermoresponsive polymer composition containing one or more EPO polypeptides) can be administered to a mammal (e.g., a human) to increase the number of red blood cells within the mammal (e.g., to increase the number of red blood cells at around, and/or near the site of administration). For example, a thermoresponsive composition described herein can be administered to a mammal to increase the number of red blood cells within the mammal by, for example, 10, 20, 30, 40, 50, 60, 70, 80, 90, 95, or more percent.

In some cases, a thermoresponsive composition described herein (e.g., a thermoresponsive polymer composition containing one or more EPO polypeptides) can be administered to a mammal (e.g., a human) to increase the number of local blood vessels (e.g., to promote neovascularization such as angiogenesis at, around, and/or near the site of administration) within the mammal. For example, a thermoresponsive composition described herein can be administered to a mammal to increase the number of blood vessels within the mammal at, around, and/or near the site of administration by, for example, 10, 20, 30, 40, 50, 60, 70, 80, 90, 95, or more percent.

In some cases, a thermoresponsive composition described herein (e.g., a thermoresponsive polymer composition containing one or more EPO polypeptides) can be administered to a mammal (e.g., a human) having a nerve injury to improve the function (e.g., motor function and/or sensory function) of the injured nerve. For example, a thermoresponsive composition described herein can be administered to a mammal having a nerve injury to improve the function of the injured nerve by, for example, 10, 20, 30, 40, 50, 60, 70, 80, 90, 95, or more percent.

In some cases, a thermoresponsive composition described herein (e.g., a thermoresponsive polymer composition containing one or more EPO polypeptides) can be administered to a mammal (e.g., a human) having a nerve injury to reduce or eliminate one or more symptoms of the nerve injury (e.g., pain, sensitivity, numbness, tingling, prickling, burning, problems with positional awareness, loss in grip strength, and/or difficulty walking). For example, a thermoresponsive composition described herein can be administered to a mammal having a nerve injury to reduce or eliminate one or more symptoms of a nerve injury by, for example, 10, 20, 30, 40, 50, 60, 70, 80, 90, 95, or more percent.

In some cases, a thermoresponsive composition described herein (e.g., a thermoresponsive composition containing one or more EPO polypeptides) can be administered to a mammal (e.g., a human) having a wound (e.g., a skin wound or an internal wound) to accelerate wound healing within the mammal. For example, a thermoresponsive composition described herein can be administered to a mammal having a wound to accelerate wound healing within the mammal by, for example, 10, 20, 30, 40, 50, 60, 70, 80, 90, 95, or more percent.

In some cases, a thermoresponsive composition described herein (e.g., a thermoresponsive composition containing one or more EPO polypeptides) can be administered to a mammal (e.g., a human) having a wound (e.g., a skin wound or an internal wound) to reduce or eliminate the breakdown of anastomosis within the mammal. For example, a thermoresponsive composition described herein can be administered to a mammal having a wound to reduce the breakdown of anastomosis within the mammal by, for example, 10, 20, 30, 40, 50, 60, 70, 80, 90, 95, or more percent.

In some cases, a thermoresponsive composition described herein (e.g., a thermoresponsive composition containing one or more EPO polypeptides) can be administered to a mammal (e.g., a human) having a wound (e.g., a skin wound or an internal wound) to accelerate the onset of an inflammatory phase of wound healing. For example, a thermoresponsive composition described herein can be administered to a mammal having a wound to accelerate the onset of an inflammatory phase of wound healing within the mammal by, for example, 10, 20, 30, 40, 50, 60, 70, 80, 90, 95, or more percent.

In some cases, a thermoresponsive composition described herein (e.g., a thermoresponsive composition containing one or more EPO polypeptides) can be administered to a mammal (e.g., a human) having a wound (e.g., a skin wound or an internal wound) to increase neovascularization (e.g., angiogenesis) at the wound. For example, a thermoresponsive composition described herein can be administered to a mammal having a wound to increase neovascularization (e.g., angiogenesis) at the wound within the mammal by, for example, 10, 20, 30, 40, 50, 60, 70, 80, 90, 95, or more percent.

Any appropriate mammal having a nerve injury and/or having a wound (e.g., a skin wound or an internal wound) can be treated as described herein (e.g., by administering a thermoresponsive composition described herein). Examples of mammals that can have a nerve injury and/or a wound and that can be treated as described herein include, without limitation, humans, non-human primates such as monkeys, horses, bovine species, porcine species, dogs, cats, mice, rats, rabbits, and goats. In some cases, a human having a nerve injury can be treated as described herein. In some cases, a human having a wound (e.g., a skin wound or an internal wound) can be treated as described herein.

A mammal having any type of nerve injury can be treated as described herein (e.g., by administering a thermoresponsive composition described herein). Examples of types of nerve injuries that can be treated as described herein include, without limitation, peripheral nerve injuries, traumatic nerve injuries, crush injuries, stretched nerves, pinched nerves, compressed nerves, pinched nerves, wounds (e.g., skin wounds), somatic nerve injury in the gut, spinal nerve root injury, and cauda equine. In some cases, an injured nerve that can be treated as described herein is not a transected nerve. In some cases, an injured nerve that can be treated as described herein is not a severed nerve. In some cases, an injured nerve can be associated with a disease or disorder such as carpel tunnel syndrome, sciatica, a diabetic wound, ileus, or a pressure sore.

A nerve injury that can be treated as described herein (e.g., by administering a thermoresponsive composition described herein) can affect any type of nerve. In some cases, an injured nerve that can be treated as described herein can be a sensory neuron. In some cases, an injured nerve that can be treated as described herein can be a motor neuron. In some cases, an injured nerve that can be treated as described herein can be a peripheral nerve (e.g., a somatic nerve). In some cases, an injured nerve that can be treated as described herein can be in a nerve plexus. Examples of types of nerves that can be injured and can be treated as described herein include, without limitation, the sciatic nerve, ulnar nerve, radial nerve, median nerve, femoral nerve, a somatic nerve in the gut, a somatic nerve in the skin, a spinal root nerve, peripheral nerves in the limbs and/or plexi, nerves in the gut, and nerves in the skin.

A nerve injury that can be treated as described herein (e.g., by administering a thermoresponsive composition described herein) can affect a nerve in any location within a mammal. In some cases, an injured nerve that can be treated as described herein can be in the brain of a mammal. In some cases, an injured nerve that can be treated as described herein can be in the spinal cord of a mammal.

In some cases, methods described herein also can include identifying a mammal as having a nerve injury. Examples of methods for identifying a mammal as having a nerve injury include, without limitation, physical examinations, electromagnetic fields, functional nerve electrical stimulation techniques, electromyography, nerve conduction studies, and magnetic resonance imaging. Once identified as having a nerve injury, a mammal can be administered or instructed to self-administer a thermoresponsive composition described herein (e.g., a thermoresponsive polymer composition containing one or more EPO polypeptides).

A mammal having any type of wound can be treated as described herein (e.g., by administering a thermoresponsive composition described herein). A wound can affect any part of a mammal (e.g., any part of a mammal's body). In some cases, a wound can be a cutaneous wound or skin wound. In some cases, a wound can be an internal wound (e.g., a wound involving the abdomen, a muscle, and/or a vessel). In some cases, a wound can be a soft tissue wound (e.g., a rupture of a soft tissue). In some cases, a wound can include an anastomosis (e.g., an abdominal anastomosis or a vessel anastomosis). In some cases, a wound can include a nerve injury. In some cases, a wound can be a wound that does not include a nerve injury. Examples of wounds that can be treated as described herein include, without limitation, after-shave skin wounds, abrasion skin wounds, diabetic skin wounds, bed sores, surgical wounds, anastamotic leaks, tendon gaps, muscle defects, multi-tissue disruptions (e.g., tissue disruptions involving distinct layers), and ulcerations (e.g., internal and external ulcerations) such as scrotal ulcerations, buccal sores, and gastrointestinal tract ulcerations.

In some cases, the methods described herein can include identifying a mammal (e.g., a human) as having a wound (e.g., a skin wound or an internal wound). Any appropriate method can be used to identify a mammal as having a wound. For example, visual inspection, physical examinations (e.g., for joint tenderness, swelling, redness, and flexibility), and/or imaging tests (e.g., X-rays and magnetic resonance imaging (MRI), ultrasound, computed tomography (e.g., computed tomography with or without angiography), radiographic angiography, and/or magnetic resonance angiography) can be used to identify mammals (e.g., humans) as having a wound.

A thermoresponsive composition described herein (e.g., a thermoresponsive polymer composition containing one or more EPO polypeptides) can be administered to a mammal in need thereof (e.g., a mammal having a nerve injury and/or having a wound such as a skin wound or an internal wound) at any appropriate time. In some cases, a thermoresponsive composition described herein can be administered within two weeks of a mammal sustaining a nerve injury. For example, a thermoresponsive composition described herein can be administered within from about one hour to about two weeks of the mammal sustaining a nerve injury. In some cases, a thermoresponsive composition described herein can be administered within two weeks of the mammal being identified as having a nerve injury. For example, a thermoresponsive composition described herein can be administered immediately after injury to within about two weeks of the mammal being identified as having a nerve injury. In some cases, a thermoresponsive composition described herein can be administered within two weeks of a mammal sustaining a wound (e.g., a skin wound or an internal wound). For example, a thermoresponsive composition described herein can be administered within from about one hour to about ten days of the mammal sustaining a wound. In some cases, a thermoresponsive composition described herein can be administered within two weeks of the mammal being identified as having a wound. For example, a thermoresponsive composition described herein can be administered immediately after sustaining a wound to within about two weeks of the mammal being identified as having a wound.

A thermoresponsive composition described herein (e.g., a thermoresponsive polymer composition containing one or more EPO polypeptides) can be administered to a mammal in need thereof (e.g., a mammal having a nerve injury and/or having a wound such as a skin wound or an internal wound) by any appropriate route. In some cases, a thermoresponsive composition described herein can be locally administered to a mammal in need thereof. For example, a thermoresponsive composition described herein can be administered by injecting the thermoresponsive composition (e.g., in a liquid phase) onto, into, around, and/or near an injury site nerve (e.g., a nerve injury site and/or a wound site) within a mammal (e.g., a human). For example, a thermoresponsive composition described herein can be injected as a liquid onto, into, around, and/or near an injured nerve within a mammal, and can form a gel in situ (e.g., to maintain close proximity between the thermoresponsive composition and the injury site). In some cases, the injection can be applied directly onto an open injury site (e.g., an open wound, and a surgical opening), such that the thermoresponsive composition can form a gel at an injury site. In some cases, the injection can be into the vascular (e.g., arterial) supply of a tissue having an injury site (e.g., having a nerve injury site and/or having a wound site) within a mammal (e.g., a human) such that the thermoresponsive composition can travel to the injury site via the vasculature in a liquid phase and can form a gel at the injury site. In some cases, the injection can be an image-guided injection, where an imaging technique is used to visualize the placement of one or more injection needles. Imaging techniques that can be used in image-guided injection include, without limitation, ultrasound, radiography, X-ray, CT (e.g., single-photon emission CT), fluoroscopy, positron emission tomography, and MRI. When injecting a thermoresponsive composition described herein near an injury site, the thermoresponsive composition can be injected within from about 0 mm (e.g., directly abutting an injury site) to about 5 mm (e.g., from about 0 mm to about 4 mm, from about 0 mm to about 3 mm, from about 0 mm to about 2 mm, from about 0 mm to about 1 mm, from about 1 mm to about 5 mm, from about 2 mm to about 5 mm, from about 3 mm to about 5 mm, from about 4 mm to about 5 mm, from about 1 mm to about 4 mm, from about 2 mm to about 3 mm, from about 1 mm to about 3 mm, or from about 2 mm to about 4 mm) of the injury site.

An effective amount (e.g., effective dose) of one or more EPO polypeptides in a thermoresponsive composition described herein (e.g., a thermoresponsive polymer composition containing one or more EPO polypeptides) can vary depending on the severity of the nerve injury and/or the wound (e.g., a skin wound or an internal wound), the route of administration, the age and general health condition of the subject, excipient usage, the possibility of co-usage with other therapeutic treatments such as use of other agents, and/or the judgment of the treating physician.

An effective amount of one or more EPO polypeptides in a thermoresponsive composition described herein (e.g., a thermoresponsive polymer composition containing one or more EPO polypeptides) can be any amount that can treat a nerve injury and/or a wound (e.g., a skin wound or an internal wound) within a mammal without producing significant toxicity to the mammal. An effective amount of one or more EPO polypeptides in a thermoresponsive composition described herein can be any appropriate amount. In some cases, an effective amount of one or more EPO polypeptides in a thermoresponsive composition described herein can be from about 0.005 milligrams per kilogram body weight (mg/kg) to about 5 mg/kg (e.g., from about 0.005 mg/kg to about 4 mg/kg, from about 0.005 mg/kg to about 3 mg/kg, from about 0.005 mg/kg to about 2 mg/kg, from about 0.005 mg/kg to about 1 mg/kg, from about 0.005 mg/kg to about 0.5 mg/kg, from about 0.005 mg/kg to about 0.1 mg/kg, from about 0.005 mg/kg to about 0.05 mg/kg, from about 0.05 mg/kg to about 5 mg/kg, from about 0.5 mg/kg to about 5 mg/kg, from about 1 mg/kg to about 5 mg/kg, from about 2 mg/kg to about 5 mg/kg, from about 3 mg/kg to about 5 mg/kg, from about 4 mg/kg to about 5 mg/kg, from about 0.05 mg/kg to about 4 mg/kg, from about 0.5 mg/kg to about 3 mg/kg, from about 1 mg/kg to about 2 mg/kg, from about 0.05 mg/kg to about 0.5 mg/kg, from about 0.5 mg/kg to about 1 mg/kg, from about 2 mg/kg to about 3 mg/kg, or from about 3 mg/kg to about 4 mg/kg). In some cases, an effective amount of one or more EPO polypeptides in a thermoresponsive composition described herein can be from about 2500 nanograms per milliliter serum (ng/mL) to about 2500000 ng/mL (e.g., from about 2500 ng/mL to about 250000 ng/mL, from about 2500 ng/mL to about 25000 ng/mL, from about 2500 ng/mL to about 20000 ng/mL, from about 2500 ng/mL to about 15000 ng/mL, from about 2500 ng/mL to about 10000 ng/mL, from about 2500 ng/mL to about 5000 ng/mL, from about 5000 ng/mL to about 2500000 ng/mL, from about 10000 ng/mL to about 2500000 ng/mL, from about 15000 ng/mL to about 2500000 ng/mL, from about 20000 ng/mL to about 2500000 ng/mL, from about 25000 ng/mL to about 2500000 ng/mL, from about 250000 ng/mL to about 2500000 ng/mL, or from about 25000 ng/mL to about 250000 ng/mL). The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the injured nerve may require an increase or decrease in the actual effective amount administered.

When a thermoresponsive composition described herein (e.g., a thermoresponsive polymer composition containing one or more EPO polypeptides) is formulated for controlled-release of EPO polypeptides from the thermoresponsive composition (e.g., when the thermoresponsive composition is in its gel phase), the thermoresponsive composition described herein can maintain an effective amount of EPO polypeptides for any appropriate amount of time (e.g., an appropriate amount of time after administering the thermoresponsive composition to a mammal). For example, a thermoresponsive composition described herein can maintain an effective amount of EPO polypeptides in a mammal for about 1 minute to about 4 weeks (e.g., from about 1 minute to about 3 weeks, from about 1 minute to about 2 weeks, from about 60 minutes to about 4 weeks, from about 1 hour to about 4 weeks, from about 1 day to about 4 weeks, from about 1 week to about 4 weeks, from about 1 day to about 3 weeks, from about 1 week to about 2 weeks, or from about 2 weeks to about 4 weeks) after administering the thermoresponsive composition to the mammal (e.g., after the thermoresponsive composition has formed a gel).

The frequency of administration of a thermoresponsive composition described herein (e.g., a thermoresponsive polymer composition containing one or more EPO polypeptides) can be any frequency that can treat a nerve injury and/or a wound (e.g., a skin wound or an internal wound) within a mammal without producing significant toxicity to the mammal. For example, the frequency of administration can be from about once a week to about once every two months, from about once every two weeks to about once every six weeks, or from about once every three weeks to about once a month (e.g., once every four weeks). The frequency of administration can remain constant or can be variable during the duration of treatment. A course of treatment with a thermoresponsive composition described herein can include rest periods. For example, a thermoresponsive composition described herein can be administered once a month over a six-month period followed by a rest period (e.g., a one or two month rest period), and such a regimen can be repeated multiple times. In some cases, a thermoresponsive composition described herein (e.g., a thermoresponsive polymer composition containing one or more EPO polypeptides) can be administered no more than one time to treat a nerve injury within a mammal (e.g., a human). As with the effective amount, various factors can influence the actual frequency of administration used for a particular application. For example, the effective amount, duration of treatment, use of multiple treatment agents, route of administration, and severity of the injured nerve and/or the wound may require an increase or decrease in administration frequency.

An effective duration for administering a thermoresponsive composition described herein (e.g., a thermoresponsive polymer composition containing one or more EPO polypeptides) can be any duration that treats an injured nerve and/or a wound (e.g., a skin wound or an internal wound) within a mammal without producing significant toxicity to the mammal. For example, the effective duration can vary from several days to several weeks, months, or years. In some cases, the effective duration for the treatment of an injured nerve can range in duration from about one month to about 6 months. Multiple factors can influence the actual effective duration used for a particular treatment. For example, an effective duration can vary with the frequency of administration, effective amount, use of multiple treatment agents, route of administration, and severity of the injured nerve and/or the wound being treated.

In some cases, methods described herein also can include administering to a mammal (e.g., a mammal having a nerve injury) one or more additional agents used to treat a nerve injury (e.g., one or more agents in addition to a thermoresponsive composition described herein such as a thermoresponsive composition containing one or more EPO polypeptides). The one or more additional agents used to treat an injured nerve can include any appropriate agent(s) used to treat a nerve injury. In some cases, an agent that can be used to treat a nerve injury can be an anticonvulsant. In some cases, an agent that can be used to treat a nerve injury can be a steroid (e.g., a corticosteroid and a glucocorticoid). In some cases, an agent that can be used to treat a nerve injury can be an anti-inflammatory agent (e.g., a non-steroidal anti-inflammatory drug (NSAID)). Examples of agents that can be used to treat a nerve injury include, without limitation, lamotrigine, gabapentin, valproic acid, topiramate, famotodine, phenobarbital, diphenylhydantoin, phenytoin, mephenytoin, ethotoin, mephobarbital, primidone, carbamazepine, ethosuximide, methsuximide, phensuximide, trimethadione, benzodiazepine, phenacemide, acetazolamide, progabide, clonazepam, divalproex sodium, magnesium sulfate injection, metharbital, paramethadione, phenytoin sodium, valproate sodium, clobazam, sulthiame, dilantin, diphenylan, L-5-hydroxytrytophan, methylprednisolone, dexamethasone, prednisone, salicylates (e.g., aspirin), propionic acid derivatives (e.g., ibuprofen or naproxen), acetic acid derivatives (e.g., indomethacin), oxicam derivatives (e.g., piroxicam), and fenamates (e.g., menafemic acid). In cases where a mammal having a nerve injury is treated with a thermoresponsive composition described herein and is treated with one or more additional agents used to treat a nerve injury, the additional agent(s) used to treat a nerve injury can be administered at the same time or independently. For example, the additional agent(s) used to treat a nerve injury can be formulated into a thermoresponsive composition containing one or more EPO polypeptides to form a single composition. In some cases, a thermoresponsive composition described herein can be administered first, and the one or more additional agents used to treat a nerve injury can be administered second, or vice versa.

In some cases, the severity of the nervy injury present within a mammal and/or the severity of one or more symptoms of the nerve injury being treated can be monitored. Any appropriate method can be used to determine whether or not the severity of the nervy injury present within a mammal and/or the severity of one or more symptoms of the nerve injury is reduced. For example, physical examinations, electromagnetic fields, functional nerve electrical stimulation techniques, electromyography, nerve conduction studies, and/or magnetic resonance imaging can be used to assess the severity of the nervy injury present within a mammal and/or the severity of one or more symptoms of the nerve injury.

In some cases, methods described herein can include administering to a mammal (e.g., a mammal having a skin wound) one or more additional agents used to treat a wound (e.g., one or more agents in addition to a thermoresponsive composition described herein such as a thermoresponsive composition containing one or more EPO polypeptides). The one or more additional agents used to treat a wound (e.g., a skin wound or an internal wound) can include any appropriate agent(s) used to treat a wound. In some cases, an agent that can be used to treat a wound can be an antibiotic. In some cases, an agent that can be used to treat a wound can be bacteriostatic. In some cases, an agent that can be used to treat a wound can be an antiseptic. In some cases, an agent that can be used to treat a wound can be an antimycotic. Examples of agents that can be used to treat a wound include, without limitation, penicillin, erythromycin, amoxicillin, methicillin, chlorhexidine gluconate, chloroxylenol, hydrogen peroxide, iodine, and silver (e.g., nanocrystalline silver, silver sulfadiazine, colloidal silver). In cases where a mammal having a wound is treated with a thermoresponsive composition described herein and is treated with one or more additional agents used to treat a wound, the additional agent(s) used to treat a wound can be administered at the same time or independently. For example, the additional agent(s) used to treat a wound can be formulated into a thermoresponsive composition containing one or more EPO polypeptides to form a single composition. In some cases, a thermoresponsive composition described herein can be administered first, and the one or more additional agents used to treat a wound can be administered second, or vice versa.

In some cases, the healing of the wound (e.g., a skin wound or an internal wound) present within a mammal being treated can be monitored. Any appropriate method can be used to determine the healing of the wound present within a mammal. For example, visual inspection, physical examinations (e.g., for joint tenderness, swelling, redness, and flexibility), and/or imaging tests (e.g., X-rays, MRI, ultrasound, computed tomography (e.g., computed tomography with or without angiography), radiographic angiography, and/or magnetic resonance angiography) can be used to assess the healing of the wound present within a mammal.

The invention will be further described in the following examples, which do not limit the scope of the invention described in the claims.

### EXAMPLES

### Example 1: Physiochemical Characterization of (EPO)-PLGA-PEG

### Methods

### In Vitro Release of Erythropoietin from PLGA-PEG Thermogels

Polymer solutions containing varying concentrations of EPO (0.1 U/μL, 0.25 U/μL, or 0.5 U/μL) were transferred to 1.5 mL test tubes and the samples were incubated in a water bath at 37° C. to convert them to physical hydrogels. Next, PBS (1×, pH 7.4) was added to each test tube as release media and the samples were left in the water bath at 37° C. for 28 days. At designated time points, release media was extracted from the tubes and replaced with the same amount of fresh PBS to maintain the sink condition. The samples were stored at −20° C. until analyzed. The amount of EPO in the collected supernatant was measured via enzyme-linked immunosorbent (ELISA) assay (Invitrogen Human EPO ELISA Kit) according to the manufacturer's instructions.

### Rheological Characterization of Block Copolymer Aqueous Solutions

Small amplitude oscillatory shear experiments were performed in a Discovery Hybrid Rheometer (DHR-3) from TA Instruments (New Castle, Del., USA). The rheometer was equipped with a 60 mm diameter stainless steel cone with a truncation gap of 28 μm and 1 degree cone angle. The cone was installed in the upper portion of the rheometer, which also hosted the motor and transducer for both torque and normal forces. The bottom plate was constituted by a Peltier element used to control the temperature with an accuracy of ±0.1° C.

A typical volume of 1.2 mL of solution was poured onto the bottom plate at 20° C. to minimize evaporation. The loading was followed by a trimming stage where the excess sample was removed before leading the cone to the measuring gap (μm). Once in the measuring position, the solution was surrounded by an organic non-interacting oil to avoid water evaporation. The oil viscosity was much lower than the aqueous solution so that the torque signal was largely unaffected by the presence of the oil. Rapid experiments with no oil at room temperature corroborated the results obtained with oil. Experiments conformed to previous work.

The linear viscoelastic limits were probed by means of shear strain amplitude sweep experiments at 10 rad/second and at the temperature of interest. The range of temperatures explored was 10-40° C. A shear strain of 0.03 strain units ensured the linear viscoelastic regime for the whole temperature window. Temperature sweep tests were all performed at 10 rad/second and 0.03 strain units, from 10° C. to 40° C. with a heating rate of 0.5° C./minute. The instrument accounted for thermal expansion of the measuring systems and the resulting change of gap, which served to ensure a constant measuring gap regardless of the temperature used in the experiment. The time evolution of the shear response of solutions was monitored in time sweep experiments where the frequency remained 10 rad/second and the shear strain 0.03 strain units. Frequency sweep experiments were performed at 0.03 strain units, well within the linear viscoelastic regime, at 25° C. and in the range of frequencies 100-0.1 rad/second.

The monitored rheological functions were: storage modulus G′ (elastic contribution to the material response), loss modulus G″ (the viscous contribution to the material response), and ratio G″/G′, or the loss factor tan(δ), with δ being the phase shift between the sinusoidal input exerted by the instrument and the material response output. tan(δ) was used as a parameter to detect gelation.

### Results

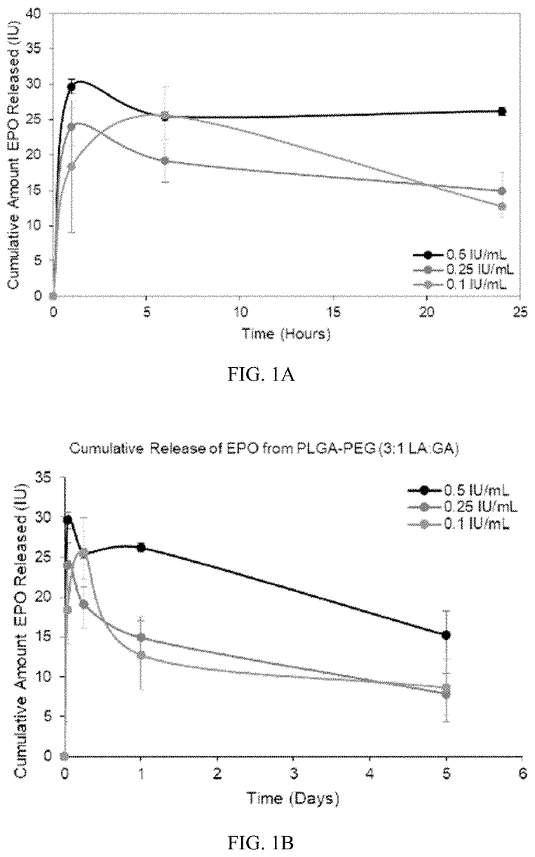

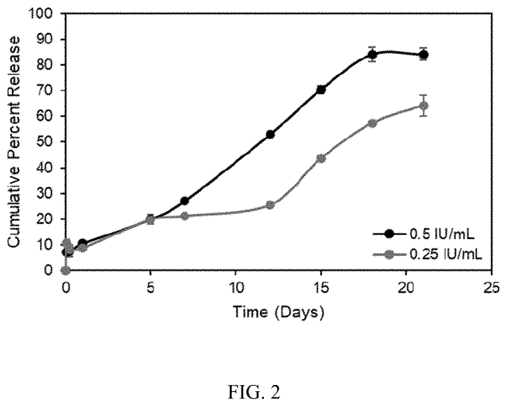

Cumulative in vitro release of EPO polypeptides from a PLGA-PEG hydrogel was examined. (EPO)-PLGA-PEG can release EPO polypeptides from the hydrogel for at least 5 days. See FIGS. 1A and 1B and FIG. 2.

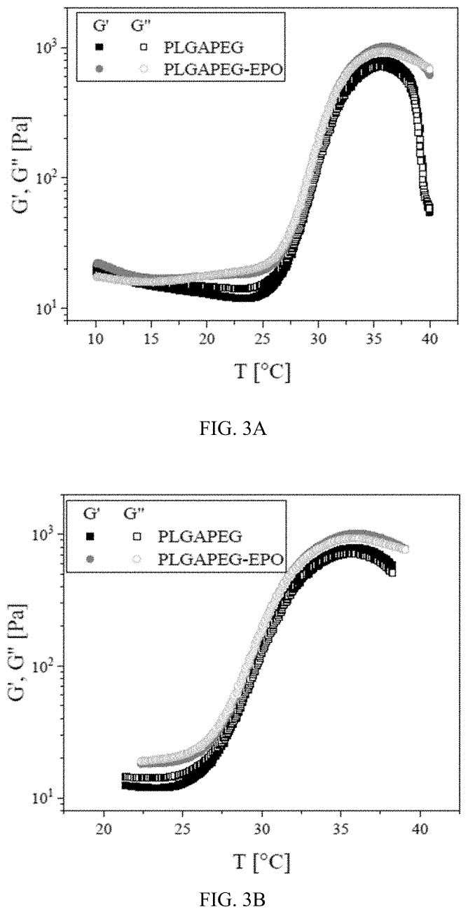

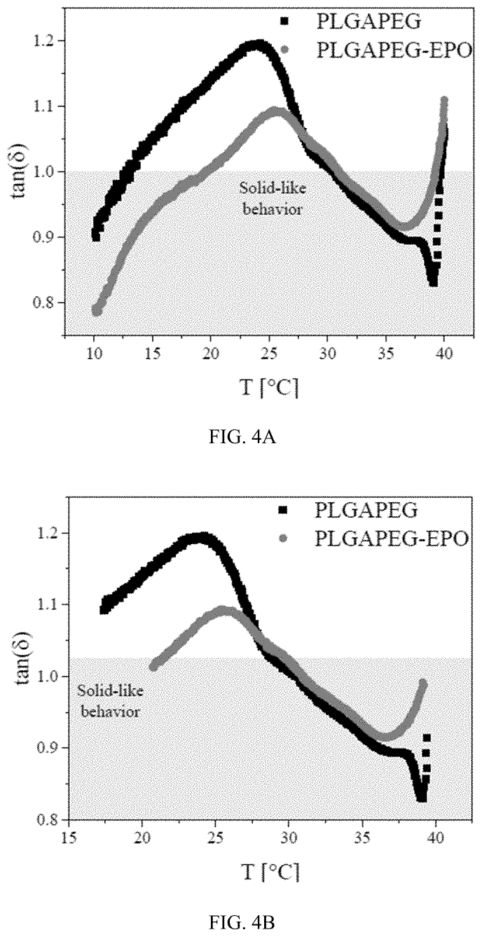

Gelation temperatures of PLGA-PEG hydrogels with or without EPO polypeptides were examined using a dynamic temperature sweep. PLGA-PEG exhibited a gelation temperature of 30.4° C. EPO-PLGA-PEG exhibited a gelation temperature of 30.9° C. Incorporation of EPO did not affect the gelation temperature. See FIGS. 3A and 3B. Tan(delta) was less than 1, indicating solid-like-behavior, for PLGA-PEG and EPO-PLGA-PEG at all temperatures above 31° C. See FIGS. 4A and 4B. This indicates that EPO-PLGA-PEG will form a gel at body temperature.

Gelation times of PLGA-PEG hydrogels with or without EPO polypeptides were also examined using a dynamic temperature sweep. PLGA-PEG gelation time was 12.8 seconds. Gelation of EPO-PLGA-PEG occurred in 16.3 seconds. Once gelation occurred, the moduli remained stable over time, indicating that EPO-PLGA-PEG forms a strong, crosslinked matrix which persists over time at body temperature.

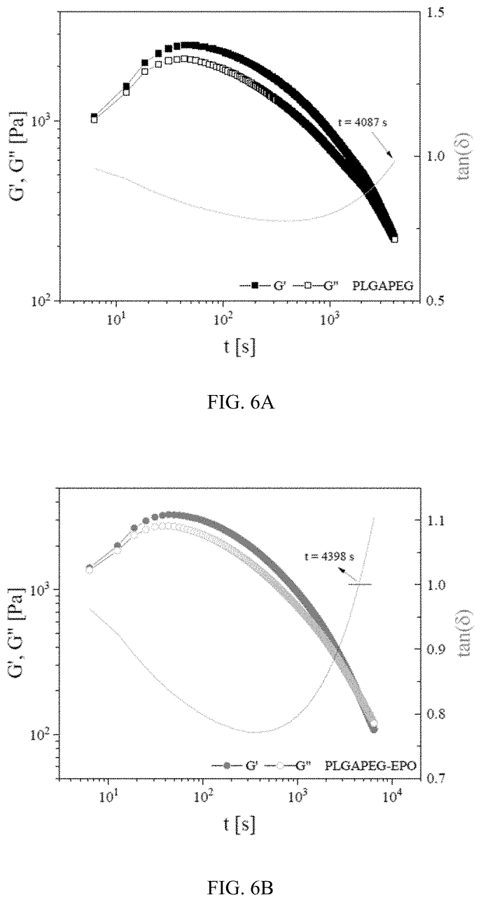

After the gel was formed and stabilized, another dynamic time sweep experiment was performed from 37° C. to 25° C. at the same frequency and strain to test the reversibility of gelation. Solution to gel (sol-gel) transition was fully reversible, going from solid to liquid phase. PLGA-PEG gel to solution transition took 4087 seconds and was fully reversible. EPO-PLGA-PEG gel to solution transition took 4398 seconds and was fully reversible. See FIGS. 6A and 6B.

### Example 2: (EPO)-PLGA-PEG as a Local Treatment for Wound Healing

### Methods

### Hematological Evaluation

Mice were anesthetized using isoflurane (IsoSol™, VEDCO) and blood samples (100 μL) were collected into K₂EDTA anticoagulant tubes (Safe-T-Fill, RAM Scientific) via retro-orbital plexus using heparinized microhematocrit capillary tubes (Fisher Scientific). Blood samples were immediately processed for hematological evaluations (Hemoglobin (Hb g/dL) and hematocrit (HCT %)) using automatic blood cell counter (Element HT5 Veterinary hematology analyzer).

### Mouse Model of Sciatic Nerve Crush Injury

Sciatic nerve crush injury was performed as previously described with pressure-gauge-tethered forceps. Briefly, after intraperitoneal (IP) ketamine (100 mg/kg)/xylazine (10 mg/kg) anesthesia, the right hindlimb was shaved and prepared with alcohol swabs and povidone-iodine (Betadine). Under a binocular microscope (Model PZMIII, World Precision Instruments), a lateral skin incision (˜2.5 cm) was made along the length of the femur and the sciatic nerve (SN) was bluntly exposed through the iliotibial band. Crush injury was performed ˜3 mm proximal to the SN trifurcation using calibrated forceps (3.3 mm tip width; 18-1107, Miltex Instruments, York, Pa.) for 30 seconds duration at a pressure of 4.4 MPa. Skin was closed via surgical staples and post-operative slow release buprenorphine (0.05 mg/kg) was given subcutaneously as an analgesic. The experimental animals (n=5/group) were randomly assigned to SN crush injury with PLGA-PEG vehicle, SN crush injury with low dose EPO-PLGA-PEG, and SN crush injury with high dose EPO-PLGA-PEG, with all treatments placed on the injury site immediately after crush injury. Post-injury functional recovery was assessed on days 1, 3, 7, 14, and 21.

### Hindlimb Grip Strength Test

A grip strength meter (BIO-GS3, Bioseb-In Vivo Research Instruments, Pinellas Park, Fla.) was used to measure hindlimb grip strength. Briefly, the mice were restrained by holding the scruff and base of the tail. Mice were allowed to hold the grid and were gently pulled along the length of the sensor grid until the grip was released. Force value was recorded 5 times per animal to calculate the average grip strength. Attention was paid to minimize paw injury and habit formation during each trial.

### Von Frey Test

Mice were placed in a transparent polycarbonate chamber (˜10×10 cm) with a metallic mesh floor approximately 25 cm above the bench. Animals were acclimatized prior to testing. SNT was performed as previously described using Von Frey filament unit (NC12775-08, Touch Test® Sensory Evaluators). Briefly, the filament pressure (1 g force) was applied to the plantar surface of the hindlimb through the mesh floor and the animal withdrawing its paw was considered a positive response. The withdrawal reflex of the hindlimb was recorded five times per animal to calculate the average percent response.

### In Vivo Degradation of EPO-PLGA-PEG and Angiogenesis in Mice

To assess in vivo biodegradation of EPO-PLGA-PEG, mice were anesthetized with intraperitoneal (IP) ketamine (100 mg/kg)/xylazine (10 mg/kg). The sciatic nerves were surgically exposed at weekly timepoints to observe location, adherence to the nerve, and mass of the gel. Images were taken with a binocular microscope to observe angiogenesis and neovascularization at and around the injury site.

### Results

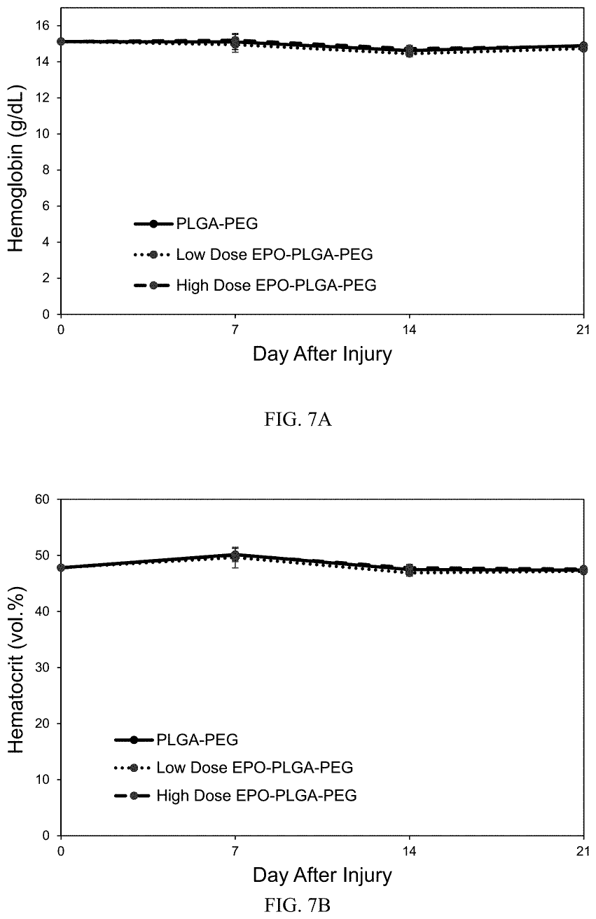

The effect of (EPO)-PLGA-PEG on hematologic parameters was examined. EPO-PLGA-PEG did not cause any adverse hematologic effects on hemoglobin (FIG. 7A) or on hematocrit (FIG. 7B) even at its peak burst release.

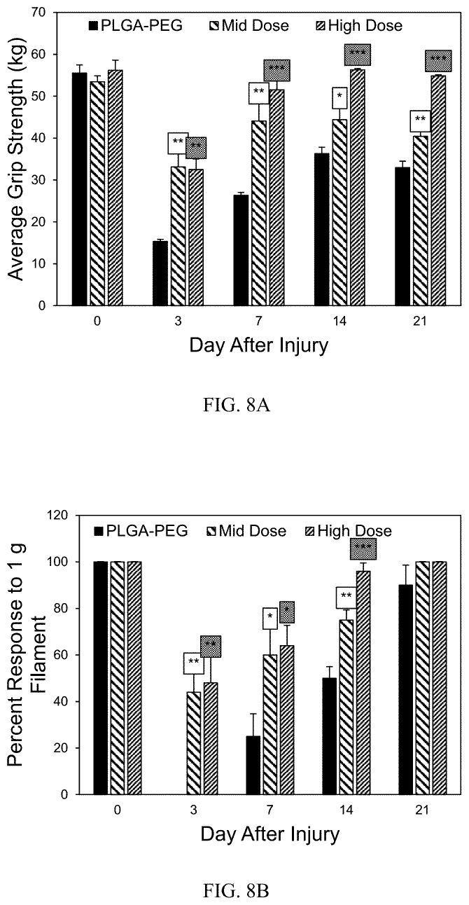

The effect of (EPO)-PLGA-PEG on motor and sensory functional recovery was examined. Animals treated with EPO-PLGA-PEG had increased grip strength relative to animals treated with PLGA-PEG (FIG. 8A). Animals treated with EPO-PLGA-PEG had response to pressure relative to animals treated with PLGA-PEG (FIG. 8B).

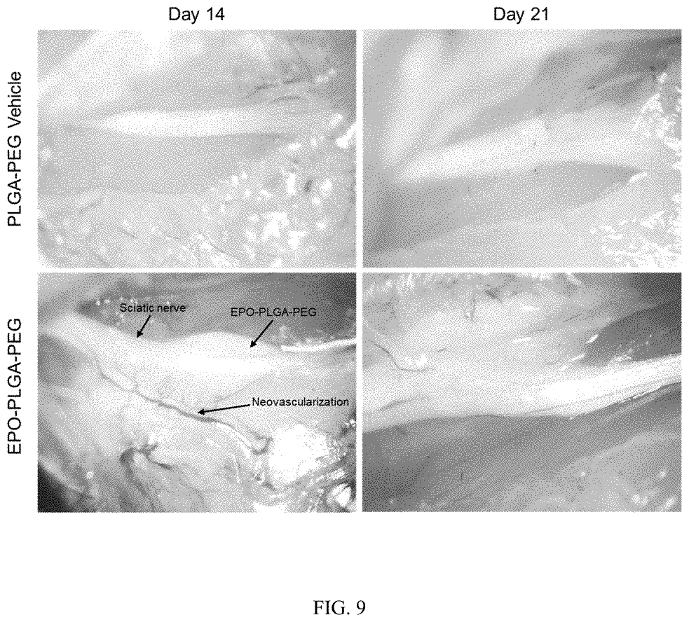

In vivo biodegradation of (EPO)-PLGA-PEG was examined. The (EPO)-PLGA-PEG biodegraded by 21 days (FIG. 9). Abundant neovascularization was also observed macroscopically at Day 14 and Day 21 post-injury.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A thermoresponsive composition comprising (a) a thermoresponsive polymer comprising poly lactic-co-glycolic acid (PLGA) and polyethylene glycol (PEG), and (b) an erythropoietin (EPO) polypeptide or a variant of said EPO polypeptide.

2. The thermoresponsive composition of claim 1, wherein said thermoresponsive composition is a liquid when below a physiological temperature of a mammal and is a gel when at or above said physiological temperature of said mammal.

3. The thermoresponsive composition of claim 2, wherein said thermoresponsive composition is a liquid at about 20° C. to about 31° C.

4. The thermoresponsive composition of claim 2, wherein said thermoresponsive composition is a gel at about 37° C.

5. The thermoresponsive composition of claim 1, wherein said PLGA has a molecular weight of about 200 Daltons (Da) to about 75,000 Da.

6. The thermoresponsive composition of claim 1, wherein said PEG has a molecular weight of about 200 Da to about 75,000 Da.

7. The thermoresponsive composition of claim 1, wherein said polymer comprises from about 0.04:1 PLGA:PEG to about 32:1 of PLGA:PEG

8. The thermoresponsive composition of claim 1, wherein said thermoresponsive composition comprises from about 0.08 M to about 4 M of said EPO polypeptide or said variant of said EPO polypeptide.

9. A method for treating a nerve injury within a mammal, wherein said method comprises administering a thermoresponsive composition comprising (a) a thermoresponsive polymer comprising poly lactic-co-glycolic acid (PLGA) and polyethylene glycol (PEG), and (b) an EPO polypeptide or a variant of said EPO polypeptide onto, into, around, and/or near said nerve injury within said mammal.

10. The method of claim 9, wherein said mammal is a human.

11. The method of claim 9, wherein said thermoresponsive composition is a liquid when below a physiological temperature of said mammal and is a gel when at or above said physiological temperature of said mammal such that said thermoresponsive composition, once administered onto, into, around, and/or near said nerve injury forms a gel in situ within said mammal.

12. The method of claim 9, wherein said administration comprises an injection.

13. The method of claim 9, wherein said gel releases about 0.005 mg/kg to about 5 mg/kg of said EPO polypeptide or said variant of said EPO polypeptide.

14. The method of claim 9, wherein said gel releases said EPO polypeptide or said variant of said EPO polypeptide for about 1 minute to about 4 weeks.

15. A method for treating a wound within a mammal, wherein said method comprises administering a thermoresponsive composition comprising (a) a thermoresponsive polymer comprising poly lactic-co-glycolic acid (PLGA) and polyethylene glycol (PEG), and (b) an EPO polypeptide or a variant of said EPO polypeptide onto, into, around, and/or near said wound.

16. The method of claim 15, wherein said mammal is a human.

17. The method of claim 15, wherein said wound is a cutaneous wound.

18. The method of claim 15, wherein said administration comprises an injection.
